(19) United States
(12) Patent Application Publication
(10) Pub. No.: US 2017/0246431 A1
(43) Pub. Date: Aug. 31, 2017

(54) MEDICAL INSTRUMENT

(71) Applicant: OLYMPUS CORPORATION, Tokyo (JP)

(72) Inventors: Takuo YOKOTA, Tokyo (JP); Masatoshi SATO, Tokyo (JP); Rei MATSUNAGA, Tokyo (JP)

(73) Assignee: OLYMPUS CORPORATION, Tokyo (JP)

(21) Appl. No.: 15/595,268

(22) Filed: May 15, 2017

Related U.S. Application Data
(63) Continuation of application No. PCT/JP2015/080205, filed on Oct. 27, 2015.

(30) Foreign Application Priority Data
Dec. 25, 2014 (JP) 2014-263293

(51) Int. Cl. A61M 25/09; A61B 1/018; A61B 17/29

(52) U.S. Cl. CPC A61M 25/09041; A61B 17/29; A61B 1/018; A61B 17/28

(57) ABSTRACT

A medical instrument, which is capable of holding a wire, includes: a sheath which has a center axis in a longitudinal direction from a distal end toward a proximal end thereof and includes in a distal end surface of the distal end a first contact area capable of being in contact with the wire and a second contact area coming in contact with the wire and provided on a proximal end side beyond the distal end with respect to the first contact area; and a holding portion which is advanceable and retractable in a lumen of the sheath and is retracted into the lumen of the sheath so that the wire is pressed to the first contact area and the second contact area and the holding portion holds the wire.

This application is a continuation application based on PCT Patent Application No. PCT/JP2015/080205, filed Oct. 27, 2015, whose priority is claimed on Japanese Patent Application No. 2014-263293, filed Dec. 25, 2014. The contents of both the PCT Patent Application and the Japanese Patent Application are incorporated herein by reference.

### BACKGROUND OF THE INVENTION

Field of the Invention

The present invention relates to a medical instrument.

Description of Related Art

In treatment or inspection of a luminal organ of a human body, it is known to introduce a medical instrument into the luminal organ using a guide wire. When an obstacle such as a stenosis or an occlusion occurs at an opening portion of the luminal organ, the guide wire itself may not be inserted into the luminal organ. For example, when a duodenal papilla is tightly closed, it is difficult to insert the guide wire into a desired luminal organ such as a bile duct or a pancreatic duct via the duodenal papilla.

As a treatment method in such a case, a method called a rendezvous method is known. In the rendezvous method, a guide wire introduced into a bile duct or a pancreatic duct from a body part other than a duodenal papilla is protruded from the duodenal papilla, and an end of the protruding guide wire is held by a medical instrument. The guide wire protruding from the duodenal papilla into a duodenum is pulled to an outside of the body through a treatment tool channel of an endoscope inserted into the duodenum. A stent placement operation or the like is performed using the guide wire pulled to the outside of the body.

When a treatment tool such as a stent is placed by the rendezvous method, the treatment tool is pushed into the bile duct or the pancreatic duct from a papilla through an endoscopic channel similarly to a procedure of ordinary endoscopic retrograde cholangiopancreatography (ERCP) or the like. However, depending on an anatomical structure of a patient, there is a case in which the duodenal papilla may not be seen from a front side in an endoscopic image or the duodenal papilla may be closed tightly. Further, there is a case in which a running state of the bile duct imagined by a surgeon may be different from a real one. In such a case, even if the surgeon attempts to push the treatment tool into the duodenal papilla by an operation at hand, the treatment tool is bent in a space between a distal end of the endoscope and the papilla and the pushing force is easily lost, and thus the treatment tool is not easily introduced.

Therefore, a method of introducing a medical instrument holding a guide wire into the bile duct or the pancreatic duct by retracting the guide wire, which is protruded from the duodenal papilla into the duodenum, into the bile duct or the pancreatic duct instead of pulling the guide wire to the outside of the body through the treatment tool channel has been proposed. A grasping forceps (for example, see Japanese Unexamined Patent Application, First Publication No. 2008-289556) is known as the medical instrument for holding the guide wire.

### SUMMARY OF THE INVENTION

According to a first aspect of the present invention, a medical instrument, which is capable of holding a wire, includes: a sheath which has a center axis in a longitudinal direction from a distal end thereof toward a proximal end thereof and includes in a distal end surface of the distal end a first contact area capable of being in contact with the wire and a second contact area coming in contact with the wire and provided on a proximal end side beyond the distal end with respect to the first contact area; and a holding portion which is advanceable and retractable in a lumen of the sheath and is retracted into the lumen of the sheath so that the wire is pressed to the first and second contact areas and the holding portion holds the wire. An inclined surface which is inclined with respect to the center axis is formed at the distal end of the sheath, and at least one of the first contact area and the second contact area is provided at the inclined surface.

According to a second aspect of the present invention, in the medical instrument according to the first aspect, the holding portion may be capable of holding the wire on a straight line connecting the first contact area and the second contact area when the sheath is seen from a center axis direction thereof.

According to a third aspect of the present invention, in the medical instrument according to the first aspect, the holding portion may be a two-legged forceps having one pair of grasping portions of which distal ends are disposed to be spaced apart from each other.

According to a fourth aspect of the present invention, in the medical instrument according to the first aspect, the holding portion may be a hook portion in which a distal end portion of the holding portion is capable of being hooked with and holding the wire.

According to a fifth aspect of the present invention, a medical instrument, which is capable of holding a wire, includes: a sheath which has a center axis in a longitudinal direction from a distal end thereof toward a proximal end thereof and includes in a distal end surface of the distal end a first contact area capable of being in contact with the wire and a second contact area coming in contact with the wire and provided on a proximal end side beyond the distal end with respect to the first contact area; and a holding portion which is advanceable and retractable in a lumen of the sheath and is retracted into the lumen of the sheath so that the wire is pressed to the first contact area and the second contact area and the holding portion holds the wire. A cut-out portion which is cut out along the center axis is formed in the distal end of the sheath, and at least one of the first contact area and the second contact area is provided at a bottom surface of the cut-out portion.

According to a sixth aspect of the present invention, in the medical instrument according to the fifth aspect, the first contact area may be provided at the distal end surface of the sheath. The second contact area may be provided at the bottom surface of the cut-out portion.

According to a seventh aspect of the present invention, in the medical instrument according to the fifth aspect, the holding portion may have an annular portion having a slit portion in which the wire is capable of being inserted, and a plate-shaped portion which extends in a direction of the center axis and is connected to the annular portion.

According to an eighth aspect of the present invention, a medical instrument, which is capable of holding a wire, includes: a sheath which has a center axis in a longitudinal direction from a distal end thereof toward a proximal end thereof and includes in a distal end surface of the distal end a first contact area capable of being in contact with the wire and a second contact area coming in contact with the wire and provided on a proximal end side beyond the distal end with respect to the first contact area; a holding portion which is advanceable and retractable in a lumen of the sheath and is retracted into the lumen of the sheath so that the wire is pressed to the first contact area and the second contact area and the holding portion holds the wire; and a lumen which is formed along the lumen of the sheath and is different from the lumen of the sheath.

### BRIEF DESCRIPTION OF THE DRAWINGS

FIG. 1 is an overall view showing a medical instrument according to a first embodiment of the present invention.

FIG. 2 is an overall view showing a state in which the medical instrument of FIG. 1 holds a guide wire.

FIG. 3 is a schematic diagram in a cross section of the medical instrument of FIG. 2 taken along a longitudinal direction thereof.

FIG. 4 is a schematic diagram when the medical instrument of FIG. 1 is seen from a center axis direction thereof.

FIG. 5 is an explanatory diagram showing a method of introducing the medical instrument according to the first embodiment of the present invention.

FIG. 6 is an explanatory diagram showing the method of introducing the medical instrument according to the first embodiment of the present invention.

FIG. 7 is an explanatory diagram showing the method of introducing the medical instrument according to the first embodiment of the present invention.

FIG. 8 is an explanatory diagram showing the method of introducing the medical instrument according to the first embodiment of the present invention.

FIG. 9 is an explanatory diagram showing the method of introducing the medical instrument according to the first embodiment of the present invention.

FIG. 10 is an explanatory diagram showing the method of introducing the medical instrument according to the first embodiment of the present invention.

FIG. 11 is an explanatory diagram showing the method of introducing the medical instrument according to the first embodiment of the present invention.

FIG. 12 is an explanatory diagram showing the method of introducing the medical instrument according to the first embodiment of the present invention.

FIG. 13 is a diagram showing a modified example of a sheath of the medical instrument according to the first embodiment of the present invention.

FIG. 14 is an overall view showing a medical instrument of a second embodiment according to the present invention.

FIG. 15 is an overall view showing a state where the medical instrument of FIG. 14 holds the guide wire.

FIG. 16 is a schematic diagram in a cross section of the medical instrument of FIG. 15 taken along a longitudinal direction thereof.

FIG. 17 is a diagram showing a modified example of a cut-out portion of the medical instrument according to the second embodiment of the present invention.

FIG. 18 is a diagram showing a modified example of the cut-out portion of the medical instrument according to the second embodiment of the present invention.

FIG. 19 is an overall view showing a medical instrument according to a third embodiment of the present invention.

FIG. 20 is an overall view showing a state where the medical instrument of FIG. 17 holds the guide wire.

FIG. 21 is an overall view showing a modified example of a holding portion according to the present invention.

FIG. 22 is an overall view showing a modified example of a medical instrument according to the present invention.

FIG. 23 is an overall view showing a state where the medical instrument of FIG. 20 holds the guide wire.

### DETAILED DESCRIPTION OF THE INVENTION

### First Embodiment

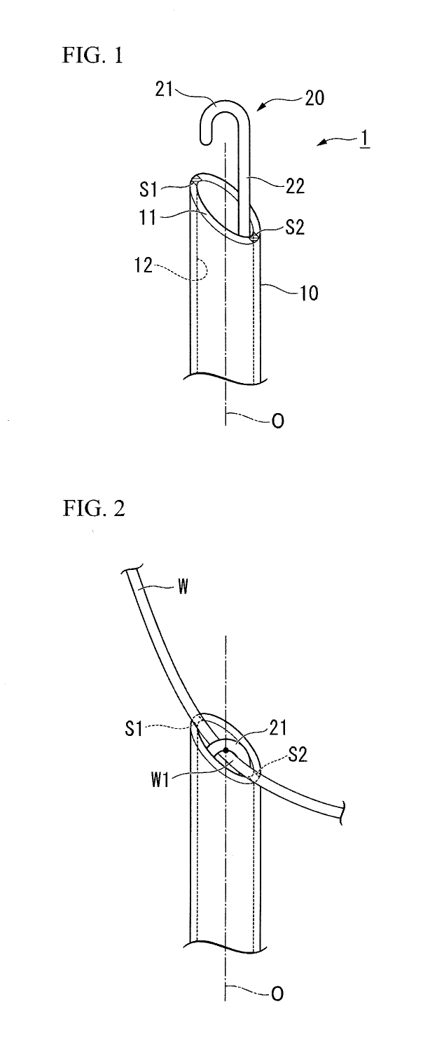

A medical instrument according to a first embodiment of the present invention will be described with reference to FIGS. 1 to 13.

A medical instrument 1 of the present invention is a medical instrument which is able to hold a wire, for example, a medical guide wire used while being inserted into a human body. The medical instrument 1 includes, as shown in FIG. 1, a sheath 10 and a hook member (a holding portion) 20.

The sheath 10 is formed in a tubular shape having a center axis O and includes a first contact area S1 which can come into contact with a guide wire to be described below and a second contact area S2 which is disposed on a proximal end side beyond the first contact area S1 and can come into contact with the guide wire. Since the sheath 10 is formed by obliquely cutting a distal end of a cylindrical tube, a distal end opening of the sheath 10 is formed in an elliptical shape. The first contact area S1 is an area on the most distal end side of a distal end surface 11 of the sheath 10, and the second contact area S2 is an area on the most proximal end side of the distal end surface 11 of the sheath 10. That is, the first contact area S1 and the second contact area S2 are disposed at positions at which phases thereof are deviated by 180° when being seen from a direction of the center axis O.

The hook member 20 is disposed in a lumen 12 of the sheath 10 to be advanceable and retractable in a direction parallel to the center axis O. Specifically, the hook member 20 includes a hook portion 21 which is formed at a distal end side thereof and a bar-shaped portion 22 which extends toward a proximal end side thereof. The hook portion 21 is configured such that the guide wire can be hooked on and held by the hook portion 21.

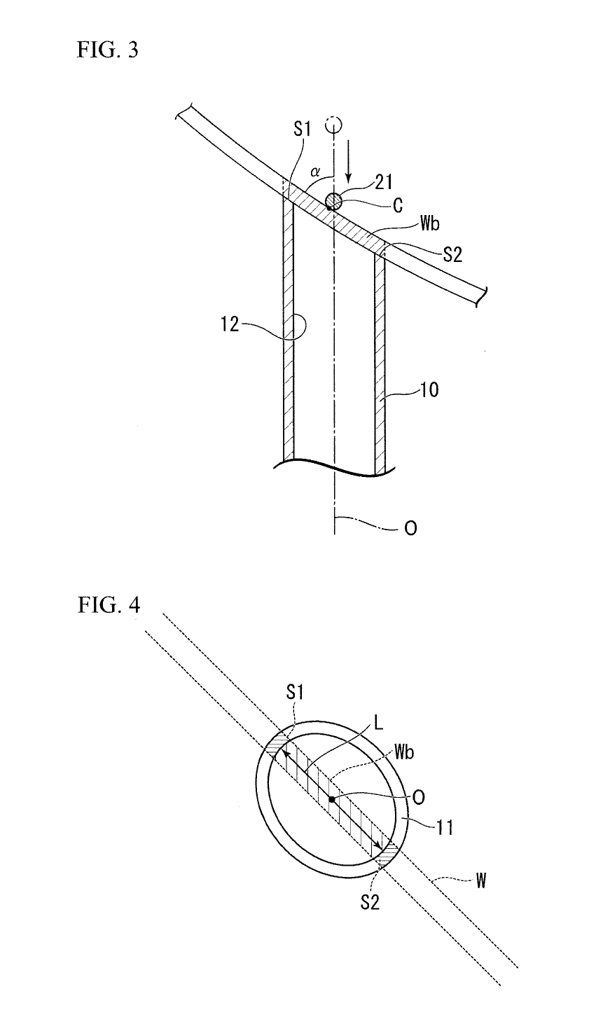

As shown in FIG. 2, when the hook member 20 is slightly retracted while the guide wire W is hooked with the hook portion 21, the guide wire W is pressed to the distal end surface of the sheath 10. As a result, the guide wire W is disposed to be in contact with the first contact area S1 and the second contact area S2. At this time, as shown in the cross section in FIG. 3, the center axis O and a guide wire Wb between the first contact area S1 and the second contact area S2 form an acute angle at a side of the first contact area S1. That is, an angle α formed between the center axis O and the guide wire Wb located on the distal end side beyond a contact portion C at which the hook portion 21 and the guide wire Wb are in contact with each other is an acute angle.

The hook member 20 can hold the guide wire Wb on a straight line L connecting the first contact area S1 with the second contact area S2 when the sheath 10 is seen from the direction of the center axis O, as shown in FIG. 4. Accordingly, the guide wire W can be kept in a state in which the guide wire W is in contact with the first contact area S1 and the second contact area S2.

Next, a method of introducing the medical instrument 1 into a luminal organ, for example, a bile duct by a rendezvous method will be described.

As the rendezvous method, there are two methods including a method of puncturing a bile duct (a first luminal organ) from an inside of the body and a method of puncturing the bile duct from an outside of the body. However, in the following description, an example of puncturing the bile duct Bd from the inside of the body is used.

[First Step]

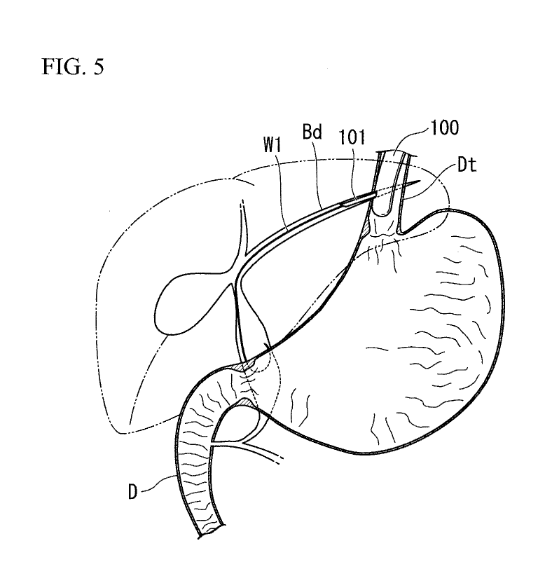

In the case of puncturing the bile duct from the inside of the body, a known ultrasound endoscope is used. First, as shown in FIG. 5, an ultrasound endoscope 100 is inserted orally into an alimentary canal Dt. Then, the bile duct Bd is checked with an ultrasound image, a puncture needle 101 is inserted into the bile duct Bd through the alimentary canal Dt, and a guide wire W1 is protruded from the puncture needle 101 and then introduced into the bile duct Bd.

[Second Step]

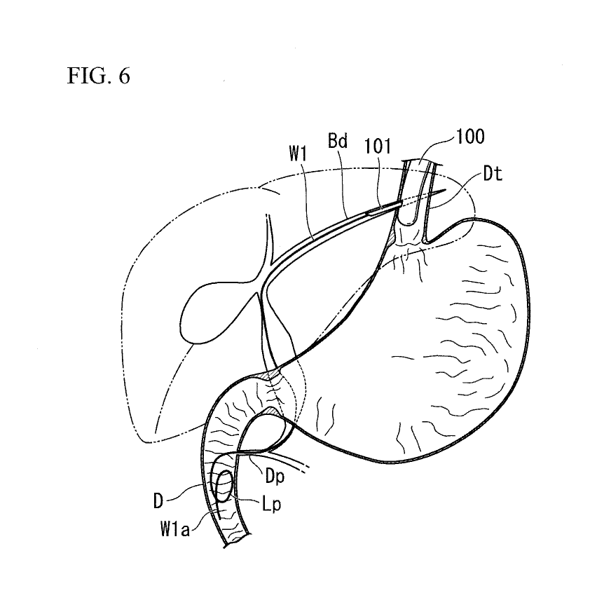

Next, as shown in FIG. 6, a surgeon pushes the first guide wire W1 inserted into the bile duct Bd forward so that a distal end W1a of the first guide wire W1 is protruded from a duodenal papilla (an opening portion) Dp into a duodenum (a second luminal organ) D. By pushing the first guide wire W1 toward the duodenal papilla Dp, the distal end W1a of the first guide wire W1 protruding from the duodenal papilla Dp generally forms a loop Lp in a lumen of the duodenum D. Then, the ultrasound endoscope 100 is removed to the outside of the body, and the distal end W1a of the first guide wire W1 is retained in the duodenum D. At this time, a proximal end side of the first guide wire W1 is located outside the patient's body.

Due to the loop Lp formed at the distal end of the first guide wire W1, the first guide wire W1 can be prevented from coming out of the body together with the ultrasound endoscope 100 when the ultrasound endoscope 100 is removed to the outside of the body. In this manner, the distal end W1a of the first guide wire W1 is retained in the duodenum D.

[Third Step]

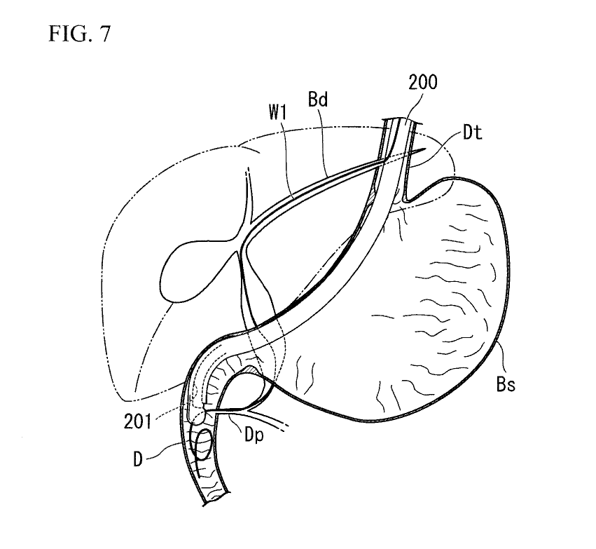

Next, as shown in FIG. 7, a side view type endoscope 200 is inserted to the vicinity of the duodenal papilla Dp via a stomach Bs. Then, the medical instrument 1 is inserted through a treatment tool channel 201 of the endoscope 200.

[Fourth Step]

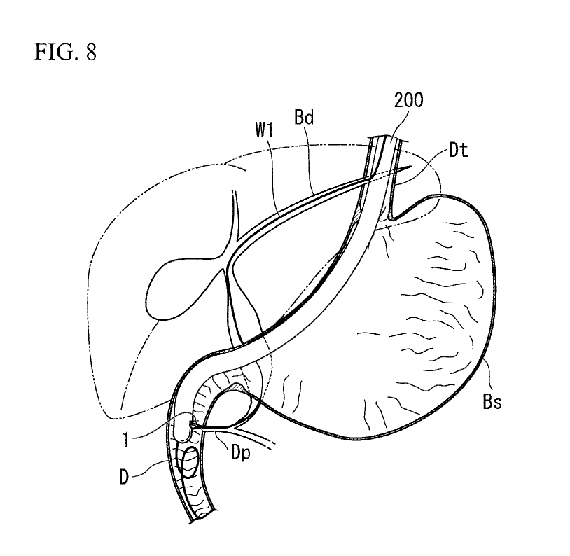

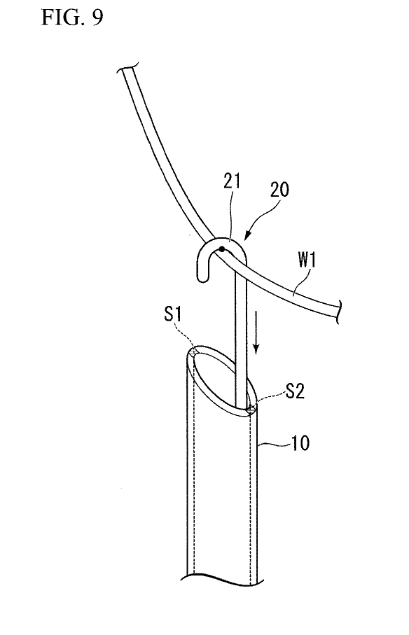

Next, as shown in FIG. 8, the hook member 20 is protruded from the sheath 10 of the medical instrument 1 while the first guide wire W1 protruding from the duodenal papilla Dp is checked with an image of the endoscope 200. Then, as shown in FIG. 9, the first guide wire W1 is hooked with the hook portion 21 of the medical instrument 1 from a side substantially perpendicular thereto. As shown in FIG. 2, the first guide wire W1 is brought into contact with the first contact area S1 and the second contact area S2 by pulling the hook member 20 into the lumen 12 of the sheath 10. The angle α formed by the wire Wb and the center axis O is an acute angle, and the entire guide wire W1 and the medical instrument 1 also form an approximate acute angle.

[Fifth Step]

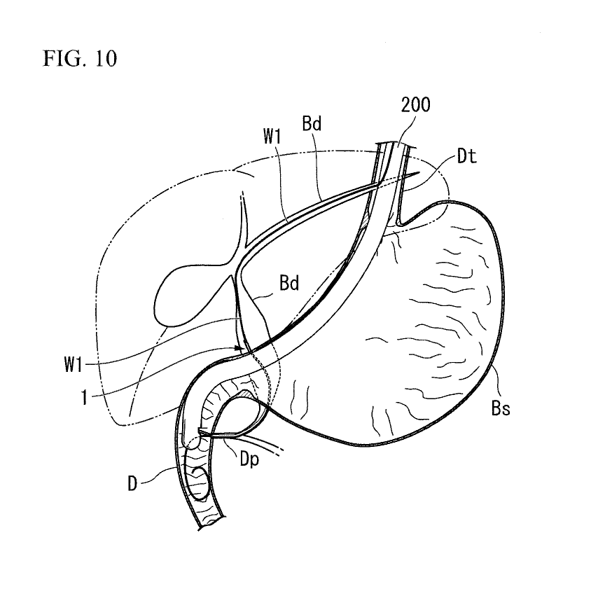

Next, the surgeon pulls the proximal end side of the first guide wire W1 outside the patient's body toward the outside of the body. As a result of this operation, as shown in FIG. 10, the medical instrument 1 is pulled into the bile duct Bd from the duodenal papilla Dp together with the first guide wire W1 while the hook member 20 of the medical instrument 1 holds the first guide wire W1. Since the guide wire W1 and the medical instrument 1 form the acute angle, the guide wire W1 and the medical instrument 1 smoothly enter the bile duct Bd from the duodenal papilla Dp. In accordance with the pulling of the first guide wire W1, the distal end of the medical instrument 1 advances toward an upstream side inside the bile duct Bd. Even at this time, as shown in FIG. 3, since the angle α formed between the center axis O and the wire Wb located on the distal end side beyond the contact area C at which the hook portion 21 and the wire Wb are in contact with each other is the acute angle, the sheath 10 is bent along an extension direction of the guide wire W. In this state, the medical instrument 1 advances inside the bile duct Bd.

[Sixth Step]

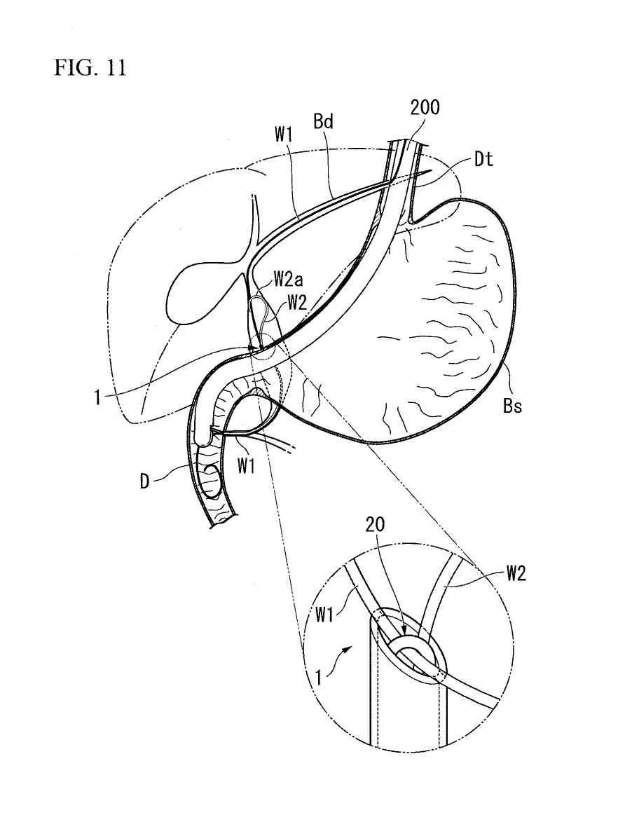

Next, the surgeon inserts a second guide wire W2 from a hand side into the sheath 10 inserted through the treatment tool channel 201 of the endoscope 200 so that the second guide wire W2 is protruded from the distal end of the sheath 10. Accordingly, as shown in FIG. 11, the second guide wire W2 is placed in the bile duct Bd from the distal end opening of the sheath 10. A distal end W2a of the second guide wire W2 also forms a loop like the first guide wire W1. At this time, a proximal end side of the second guide wire W2 is located outside the patient's body.

[Seventh Step]

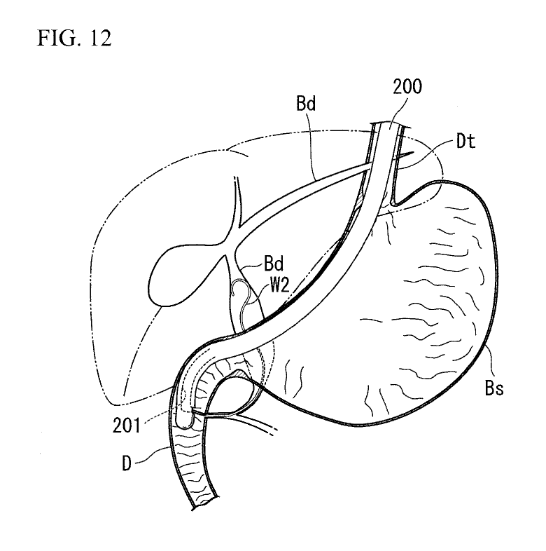

Next, the surgeon releases the holding of the first guide wire W1 by the medical instrument 1 and pulls the proximal end side of the first guide wire W1 toward the outside of the body so that the first guide wire W1 is removed to the outside of the body. Further, as shown in FIG. 12, the surgeon retracts the medical instrument 1 so that the medical instrument 1 is accommodated in the treatment tool channel 201 of the endoscope 200 and then removes the medical instrument 1 to the outside of the body through the treatment tool channel 201.

[Eighth Step]

Next, the same treatment as that in ordinary endoscopic retrograde cholangiopancreatography (ERCP) is performed using the second guide wire W2. Since a procedure of ERCP is the same as the known procedure, a description thereof will be omitted.

According to the medical instrument 1 of the present embodiment, since the sheath 10 is formed such that the center axis O and the guide wire Wb between the first contact area S1 and the second contact area S2 form the acute angle at the side of the first contact area S1 when the guide wire W1 is disposed to be in contact with the first contact area S1 and the second contact area S2, the guide wire W1 can be held while the longitudinal direction of the sheath 10 and the extension direction of the guide wire W1 are substantially parallel. Thus, even when being used in the rendezvous method, the medical instrument 1 can be easily introduced from the duodenal papilla Dp by pulling the guide wire W1. Therefore, the medical instrument 1 can be easily inserted from the duodenal papilla Dp toward the bile duct Bd even when the duodenal papilla Dp is stenosed or occluded. Further, the medical instrument 1 can be also smoothly introduced even when the duodenal papilla Dp is not stenosed.

Further, by using the sheath 10 of which the distal end is obliquely cut, the first contact area S1 and the second contact area S2 can be formed with a simply machining process, the guide wire Wb and the center axis O can form the acute angle, and the angle α can also be easily adjusted.

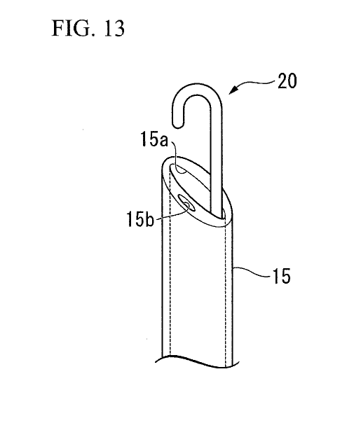

In addition, a configuration using a sheath 15 including a first lumen 15a and a second lumen 15b like a modified example shown in FIG. 13 may be used in the medical instrument of the present invention. The first lumen 15a and the second lumen 15b are disposed in parallel in the longitudinal direction.

Due to such a configuration, the hook member 20 can be inserted into the first lumen 15a, and a contrast medium can be flowed into the second lumen 15b. Therefore, a state of the bile duct can be checked by flowing the contrast medium into the bile duct when the medical instrument is introduced into the bile duct. Also, the guide wire W2 may be inserted into the second lumen 15b. Since the hook member 20 and the guide wire W2 are disposed in different lumens, interference therebetween does not occur.

Further, the first contact area S1 and the second contact area S2 have been provided at the positions at which phases thereof are deviated by 180°, but the present invention is not limited thereto. When the second contact area is disposed on the proximal end side of the sheath beyond the first contact area, the same effect can be obtained. However, when the phases are deviated by 180°, there is an advantage that the guide wire W can be easily held because the guide wire Wb passes through the center axis O.

Also, a pre-curve (a bending tendency) may be applied such that a surface of the sheath 10 including the second contact area S2 is on an inner side. In such a configuration, since the sheath 10 easily follows the guide wire W, the sheath is easily inserted into the duodenal papilla Dp. Furthermore, the sheath 10 is easily bent by applying the same bending tendency as that of the sheath 10 to the hook member 20.

### Second Embodiment

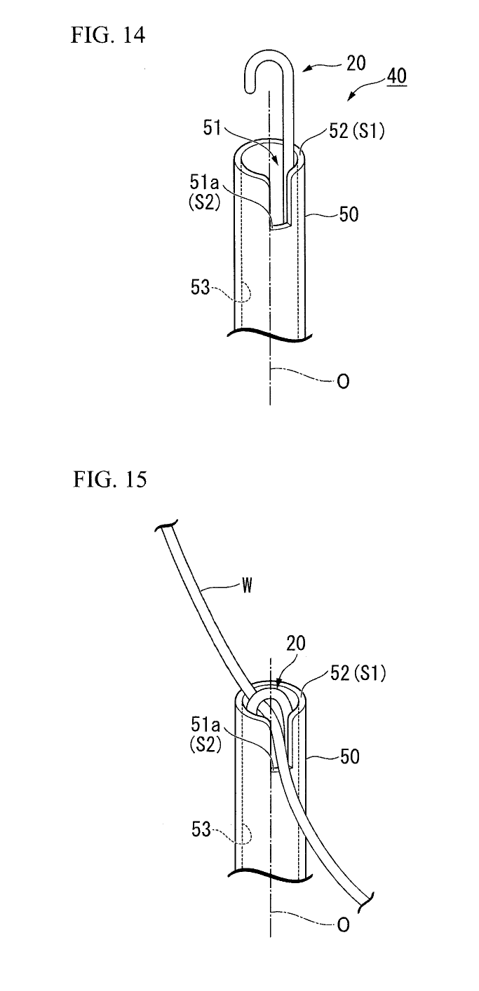

A second embodiment of the present invention will be described with reference to FIGS. 14 to 18.

A medical instrument 40 of the present embodiment is different from that of the first embodiment in a configuration of the sheath.

In the following description, the same reference numerals will be given to the same components as those of the above description, and a repetitive description thereof will be omitted.

As shown in FIG. 14, a sheath 50 has a substantially tubular shape, and a cut-out portion 51 is formed in a distal end thereof to be cut out into a rectangular shape substantially parallel to a center axis O. In the medical instrument 40, the first contact area S1 is a distal end surface 52 of the sheath 50, and the second contact area S2 is a bottom surface 51a of the cut-out portion 51.

A dimension of the cut-out portion 51 in a direction perpendicular to the center axis O is formed larger than that of an outer diameter of the guide wire W. Therefore, the guide wire W can be inserted into the cut-out portion 51.

The hook member 20 is disposed in a lumen 53 of the sheath 50 to be advanceable and retractable in a direction parallel to the center axis O.

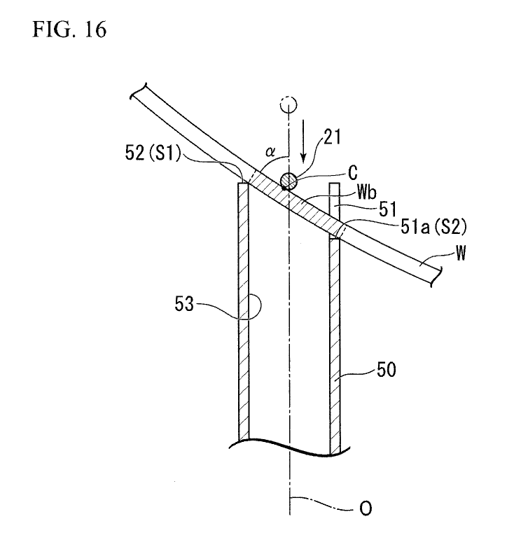

As shown in FIG. 15, when the hook member 20 is slightly retracted while the guide wire W is hooked with the hook portion 21, the guide wire W is pressed to the distal end surface of the sheath 10. As a result, the guide wire W is disposed to be in contact with the first contact area S1 disposed at the distal end surface 52 of the sheath and the second contact area S2 disposed at the bottom surface 51a of the cut-out portion 51. At this time, as shown in the cross section in FIG. 16, the center axis O and the guide wire Wb between the first contact area S1 and the second contact area S2 form an acute angle at a side of the first contact area S1. That is, an angle α formed between the center axis O and the wire Wb located on the distal end side beyond a contact area C at which the hook portion 21 and the wire Wb are in contact with each other is an acute angle.

When the medical instrument 40 is introduced into a bile duct, a method in the present embodiment is different from the method described in the first embodiment in only the fourth step.

In the present embodiment, in the fourth step, the guide wire W is hooked with the hook portion 21 of the medical instrument 40 while the guide wire W protruding from the duodenal papilla Dp is checked with an endoscopic image. Then, as shown in FIG. 16, when the hook member 20 is retracted, the guide wire W is pulled such that the guide wire W1 at a distal end side is brought into contact with the first contact area S1 and the guide wire W1 at a proximal end side is brought into contact with the second contact area S2. Accordingly, the angle α formed between the center axis O and the wire Wb located on the distal end side beyond the contact portion C at which the hook portion 21 and the guide wire Wb are in contact with each other becomes an acute angle.

According to the medical instrument 40 of the present embodiment, since the second contact area S2 is disposed at the bottom surface 51a of the cut-out portion 51, the guide wire W can be held without positional deviation from the second contact area S2.

Further, since the cut-out portion 51 is cut out toward the proximal end side of the sheath 50, it is easy to provide the first contact area S1 on the distal end side beyond the second contact area S2. Therefore, the angle α formed between the center axis O and the wire Wb located on the distal end side beyond the contact portion C at which the hook portion 21 and the wire Wb are in contact with each other easily becomes an acute angle.

In addition, in the present embodiment, only the second contact area S2 is disposed at the bottom surface 51a of the cut-out portion 51, but the present invention is not limited thereto. That is, the cut-out portion may be formed at a position at which a phase thereof is deviated from the second contact area S2 by 180° when being seen from a direction of the center axis O, and the first contact area S1 may be disposed at the bottom surface of the cut-out portion. In such a configuration, the bottom surface of the cut-out portion at a side of the first contact area S1 may be formed on the distal end side compared to the bottom surface of the cut-out portion at a side of the second contact area S2.

Also, the cut-out portion of the present embodiment may be formed in the sheath 10 of the first embodiment. That is, at least one of the first contact area and the second contact area of the sheath 10 of the first embodiment may be formed at the bottom surface of the cut-out portion. Such a configuration can further prevent the positional deviation of the guide wire W, compared to the first embodiment. Further, the angle α can be adjusted without changing the shape of the distal end surface.

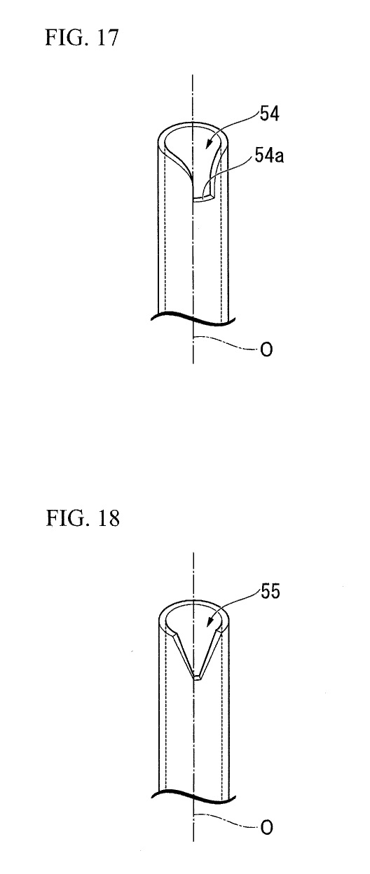

In addition, in the present embodiment, the cut-out portion 51 has been formed in the rectangular shape, but the present invention is not limited thereto. The cut-out portion 51 may be formed in a trapezoidal shape or a triangular shape which has a long side at the distal end side of the sheath 50. For example, as shown in FIG. 17, a cut-out portion 54 of which a cut-out dimension in a direction perpendicular to the center axis O is gradually reduced toward a bottom surface 54a may be used. Like this, since the distal end side of the sheath 50 is widely cut out, the guide wire W1 is easily disposed in the cut-out portion 51 when the guide wire W1 is pulled with the hook member 20.

Further, as shown in FIG. 18, a V-shaped cut-out portion 55 of which a cut-out dimension in a direction perpendicular to the center axis O is gradually reduced toward the proximal end side of the sheath 50 may be used. As described above, the positional deviation of the guide wire W can be prevented by narrowing the bottom surface 51a of the cut-out portion 51 or not providing the bottom surface.

### Third Embodiment

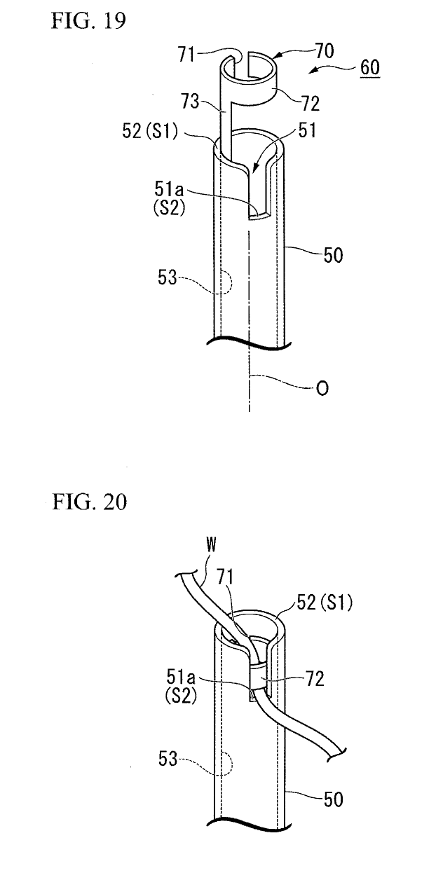

A third embodiment of the present invention will be described with reference to FIGS. 19 and 20.

A medical instrument 60 of the present embodiment is different from that of the second embodiment in a configuration of the holding portion.

In the medical instrument 60, a holding portion 70 is disposed in the lumen 53 of the sheath 50 to be advanceable and retractable in a direction parallel to the center axis O. The holding portion 70 includes an annular portion 72 having a slit portion 71 through which the guide wire W can pass and a plate-shaped portion 73 which extends in a direction of the center axis O and is connected to the annular portion 72.

A dimension of the slit portion 71 in a direction perpendicular to the center axis is formed larger than that of an outer diameter of the guide wire W. Therefore, the guide wire W can pass through the slit portion 71.

As shown in FIG. 20, the guide wire W is hooked by the slit portion 71 and disposed such that the guide wire W is in contact with the first contact area S1 and the second contact area S2. At this time, the guide wire W which is in contact with the second contact area S2 is held by the bottom surface 51a of the cut-out portion 51 and the annular portion 72. However, the annular portion 72 and the guide wire W are not in contact with each other at the first contact area S1, and the guide wire W is supported by the first contact area S1.

According to the medical instrument 60 of the present embodiment, since the guide wire W in contact with the second contact area S2 can be held such that the guide wire W is interposed between the bottom surface 51a of the cut-out portion 51 and the annular portion 72, a position of the guide wire W in contact with the second contact area S2 can be fixed. That is, since it is difficult for the guide wire W to escape from the cut-out portion 51, it is easier to introduce the medical instrument 60 from a duodenal papilla Dp.

Further, the sheath 50 of the second embodiment is used in the present embodiment. However, the holding portion 70 of the present embodiment may be used in the sheath 10 of the first embodiment.

While the preferred embodiments of the present invention have been described above, the present invention is not limited to these embodiments. Additions, omissions, substitutions, and other modifications of the configurations can be made without departing from the scope of the present invention.

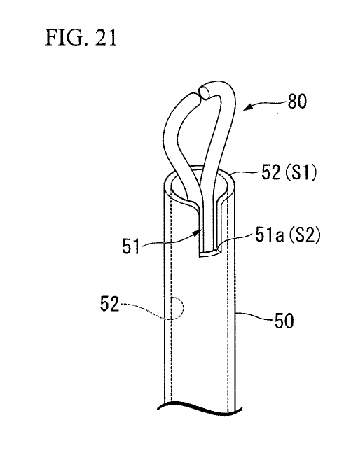

For example, as shown in FIG. 21, a two-legged forceps 80 having one pair of grasping portions of which distal ends are disposed to be spaced apart from each other may be used as the holding portion.

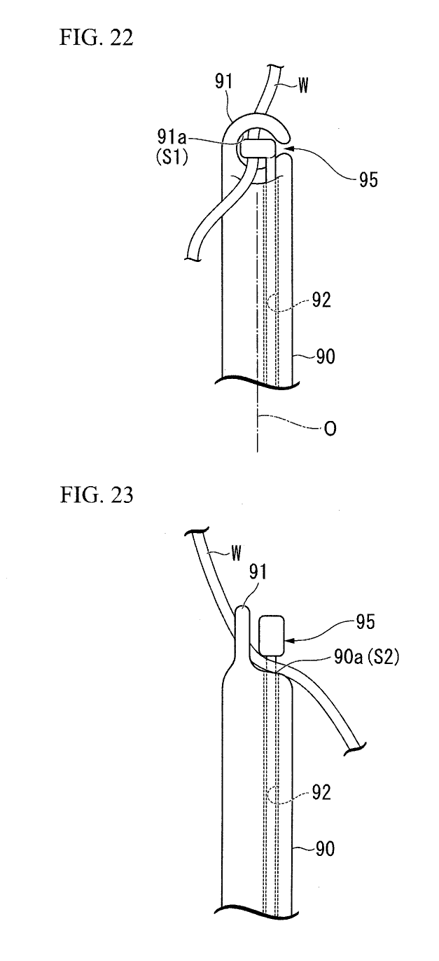

Also, as in a modified example shown in FIG. 22, a sheath 90 at which a hook potion 91 protruding toward a distal end thereof is formed may also be used. A lumen 92 is formed at the sheath 90. A holding portion 95 is disposed to be advanceable and retractable in the lumen 92. An external dimension of a distal end of the holding portion 95 is larger than an inner diameter of the lumen 92. A shape of a distal end portion of a holding portion in a modified example may be the hook portion 21 of the above-described hook member 20.

In the modified example, the first contact area S1 is an inner surface 91a of the hook portion 91, and the second contact area S2 is a distal end surface 90a of the sheath 90 as shown in FIG. 23.

Even in such a configuration, the guide wire W is held by a distal end portion of the holding portion 95 such that the guide wire W is brought into contact with the first contact area S1 and the second contact area S2, and thus the center axis O and the guide wire Wb between the first contact area S1 and the second contact area S2 form an acute angle at a side of the first contact area S1. Accordingly, it is easy to introduce the medical instrument from a duodenal papilla Dp.

[Additional Note]

The present invention includes the following technical idea.

A method of inserting a medical instrument, which includes a sheath having a center axis, a first contact area capable of being in contact with the guide wire, and a second contact area capable of being in contact with the guide wire and provided on a proximal end side beyond the first contact area, and a holding portion disposed to be advanceable and retractable in a lumen of the sheath and holding the guide wire, into a luminal organ includes: a step of inserting a distal end of the guide wire into a first luminal organ from an outside of a body; a step of causing the guide wire inserted into the first luminal organ to protrude from an opening portion of the first luminal organ into a second luminal organ communicating with the first luminal organ via the opening portion of the first luminal organ and placing a distal end part of the guide wire in the second luminal organ; a step of inserting an endoscope to the second luminal organ, inserting the medical instrument through a treatment tool channel of the endoscope, and causing the medical instrument to protrude from the endoscope; a step of holding the guide wire with the holding portion of the medical instrument such that the guide wire is in contact with the first contact area and the second contact area; and a step of pulling the guide wire to the outside of the body so that a distal end portion of the medical instrument is pulled from the second luminal organ into the first luminal organ via the opening portion.

While the preferred embodiments of the present invention have been described above, the present invention is not limited to these embodiments and modified examples thereof. It is should be understood that additions, omissions, substitutions, and other modifications of the configurations can be made without departing from the scope of the present invention.

Also, the present invention is not limited by the description above and is limited only by the appended claims.

What is claimed is:

1. A medical instrument which is capable of holding a wire, comprising:
a sheath which has a center axis in a longitudinal direction from a distal end thereof toward a proximal end thereof and includes in a distal end surface of the distal end a first contact area capable of being in contact with the wire and a second contact area coming in contact with the wire and provided on a proximal end side beyond the distal end with respect to the first contact area; and
a holding portion which is advanceable and retractable in a lumen of the sheath and is retracted into the lumen of the sheath so that the wire is pressed to the first and second contact areas and the holding portion holds the wire,
wherein an inclined surface which is inclined with respect to the center axis is formed at the distal end of the sheath, and at least one of the first contact area and the second contact area is provided at the inclined surface.

2. The medical instrument according to claim 1, wherein the holding portion is capable of holding the wire on a straight line connecting the first contact area and the second contact area when the sheath is seen from a center axis direction thereof.

3. The medical instrument according to claim 1, wherein the holding portion is a two-legged forceps having one pair of grasping portions of which distal ends are disposed to be spaced apart from each other.

4. The medical instrument according to claim 1, wherein the holding portion is a hook portion in which a distal end portion of the holding portion is capable of being hooked with and holding the wire.

5. A medical instrument which is capable of holding a wire, comprising:
a sheath which has a center axis in a longitudinal direction from a distal end thereof toward a proximal end thereof and includes in a distal end surface of the distal end a first contact area capable of being in contact with the wire and a second contact area coming in contact with the wire and provided on a proximal end side beyond the distal end with respect to the first contact area; and
a holding portion which is advanceable and retractable in a lumen of the sheath and is retracted into the lumen of the sheath so that the wire is pressed to the first contact area and the second contact area and the holding portion holds the wire,
wherein a cut-out portion which is cut out along the center axis is formed in the distal end of the sheath, and at least one of the first contact area and the second contact area is provided at a bottom surface of the cut-out portion.

6. The medical instrument according to claim 5, wherein
the first contact area is provided at the distal end surface of the sheath, and
the second contact area is provided at the bottom surface of the cut-out portion.

7. The medical instrument according to claim 5, wherein the holding portion has an annular portion having a slit portion in which the wire is capable of being inserted, and a plate-shaped portion which extends in a direction of the center axis and is connected to the annular portion.

8. A medical instrument which is capable of holding a wire, comprising:
a sheath which has a center axis in a longitudinal direction from a distal end thereof toward a proximal end thereof and includes in a distal end surface of the distal end a first contact area capable of being in contact with the wire and a second contact area coming in contact with the wire and provided on a proximal end side beyond the distal end with respect to the first contact area;
a holding portion which is advanceable and retractable in a lumen of the sheath and is retracted into the lumen of the sheath so that the wire is pressed to the first contact area and the second contact area and the holding portion holds the wire; and
a lumen which is formed along the lumen of the sheath and is different from the lumen of the sheath.
